(19) United States
(12) Patent Application Publication
(10) Pub. No.: US 2003/0212003 A1
(43) Pub. Date: Nov. 13, 2003

(54) Cathepsin Y for the development of a medicament for the treatment of pain

(76) Inventors: Hermann Lubbert, Leverkusen (DE); Peter Engels, Bergisch Gladbach (DE); Beate Schmitz, Koln (DE)

Correspondence Address: KNOBBE MARTENS OLSON & BEAR LLP, 2040 MAIN STREET, FOURTEENTH FLOOR, IRVINE, CA, 92614, US

(21) Appl. No.: 10/369,386

(22) Filed: Feb. 14, 2003

Related U.S. Application Data
(60) Provisional application No. 60/358,008, filed on Feb. 14, 2002.

(51) Int. Cl. A61K 38/05; A61K 38/04; C12Q 1/00; A61K 31/165

(52) U.S. Cl. 514/18; 514/19; 514/617; 435/4

(57) ABSTRACT

The present invention relates to the use of a polynucleotide sequence encoding Cathepsin Y or a amino acid sequence of Cathepsin Y protein for the characterization or identification of therapeutic agents for pain, the use of such sequences for the development of a medicament, and agents useful as medicaments for the treatment of pain.

### RELATED APPLICATION

This application claims priority under 35 U.S.C. § 119(e) to U.S. Provisional Application 60/358,008, filed Feb. 14, 2002.

### FIELD OF THE INVENTION

The present invention relates to the use of a polynucleotide sequence encoding Cathepsin Y or an amino acid sequence of Cathepsin Y protein for the characterization or identification of therapeutic agents for pain, the use of such sequences for the development of a medicament, and agents useful as medicaments for the treatment of pain.

### BACKGROUND OF THE INVENTION

The effective treatment of pain requires an understanding of its physiology. It is well known, however, that stimuli which activate pain receptors in one tissue may not activate pain receptors in another. For example, pricking or cutting which causes pain in skin tissue does not cause pain in the stomach or intestine. The causes of pain in skeletal muscle, joints, and arteries can also differ (Principles of Neurology 6^{th} ed. 1997 Adams, R. D. et al. eds. McGraw-Hill pp. 133-134). Consequently, methods useful for relieving one type of pain are often less effective, or even ineffective, when applied to the alleviation of others. In general, neuropathic pain is persistent and is characterized by burning, gnawing, aching, shooting, or lancinating sensations. It is frequently associated with hyperesthesia, hyperalgesia, allodynia, and hyperpathia, and in some cases by sensory deficit or autonomic dysfunction. Unfortunately, and unlike other types of pain, neuropathic-pain tends to respond poorly to analgesic medication (Principles of Neurology 6^{th} ed. 1997 Adams, R. D. et al. eds. McGraw-Hill p. 140).

Depending on the nerves involved, a particular instance of neuropathic pain can be classified as a central or peripheral neuropathy. Central neuropathies arise from spinal cord, brainstem, thalamic, and cerebral damage or disease, while peripheral neuropathies arise from damage or disease of peripheral nerves. Specific peripheral neuropathies include, but are not limited to: thoracic outlet obstruction syndromes; compression and entrapment neuropathies such as ulnar nerve palsy, carpal tunnel syndrome, peroneal nerve palsy, radial nerve palsy; and Guillain-Barre syndrome (The Merck Manual 16^{th} ed. 1992 1518-1522).

Neuropathic, or neurogenic, pain arises from the direct stimulation of nervous tissue. Neuropathic pain encompasses a wide variety of disorders involving single and multiple nerves. These include, but are not limited to, trigeminal neuralgia and disorders due to herpes zoster, diabetes, and trauma (including causalgia); spinal arachnoiditis and spinal cord injuries; and the thalamic pain syndrome of Dejerine-Roussy (Principles of Neurology 6^{th} ed. Adams, R. D. et al. eds. McGraw-Hill p. 140).

Neuropathic pain is caused by a variety of factors including, but not limited to: trauma caused by injury or surgical operation; tumors; bony hyperostosis; casts; crutches; prolonged cramped postures; hemorrhage into a nerve; exposure to cold or radiation; collagen-vascular disorders; infectious diseases such as Lyme disease and HIV; toxins such as emetine, hexobarbital, barbital, chlorobutanol, sulfonamides, phenytoin, nitrofurantoin, the vinca alkaloids, heavy metals, carbon monoxide, triorthocresylphosphate, orthodinitrophenol, and other solvents and industrial poisons; autoimmune reactions; nutritional deficiency, and vitamin B deficiency in particular; and metabolic disorders such as hypothyroidism, porphyria, sarcoidosis, amyloidosis, uremia and diabetes (The Merck Manual 16th ed. 1992 p. 1518).

Because so many causes of neuropathic pain exist, and because it tends to respond poorly to analgesic medication, the discovery of drugs that safely and effectively aid in its relief has been difficult.

Whereas acute pain is generally symptomatic of tissue damage or inflammation, and lasts only as long as the underlying cause remains, chronic pain may persist indefinitely in the absence, or after recovery of tissue pathology. It may occur as the aftermath of injury (e.g. to peripheral nerves or to the spinal cord), or in association with other disease states, such as herpes zoster or diabetes, but often it arises with no obvious cause. Chronic pain is often severe, leading to major disability and a high suicide rate, and it is common, with a prevalence of approximately 1% at the severe level. In contrast to acute pain, chronic pain is often unresponsive to conventional analgesic drugs (opiates and NSAIDs), and presents a difficult therapeutic problem, since its cause can usually not be resolved. Various other classes of drug, (e.g. tricyclic antidepressants, some anticonvulsant drugs such as gabapentin) may be effective, but the therapeutic response is very variable.

A common feature of many clinical syndromes where chronic pain develops is the existence of previous nerve damage, affecting peripheral nerves, the spinal cord or (as in stroke) the brain, and the concept of neuropathic pain (i.e. pain arising as a consequence of neuronal damage) has become accepted as the underlying cause of many different chronic pain conditions seen in the clinic. Several animal models of neuropathic pain have been developed, which mimic many aspects of the clinical condition. These include lesions of the sciatic nerve (constriction or partial section), section of spinal nerves, ischemic lesions of the spinal cord, diabetic neuropathy, etc. and such models have been subjected to detailed study of the anatomical, biochemical and physiological changes that accompany the development of the pain state.

In the last several years a number of experimental models for neuropathic pain have been developed (Bennett & Xie 1988 Pain 33:87-107; Seltzer et al. 1990 Pain 43:205-218; Kim & Chung 1992 Pain 50:355-363; DeLeo et al. 1994 Pain 56:9-16; Na et al. 1994 Neurosci Lett 177:50-52). The availability of these different models provides an opportunity to investigate mechanisms of neuropathic pain. Finding common features in different models should provide better insight into the mechanisms critical for neuropathic pain, and comparison of the models should help to understand pain development and progression.

Kim et al. compared three of the models on basis of neuropathic pain behaviors and the effects of surgical sympathectomy (Kim et al. 1997 Exp Brain Res 113:200-206). They found that the models of Bennett, Seltzer and Kim & Chung (see above) result in a very similar general pattern and time course of evoked pain behavior, whereby the Bennett model showed biggest behavioral signs for ongoing pain. The same results have been shown for the effects of sympathectomy on the behavioral signs of evoked and ongoing neuropathic pain, respectively. Thus these three models can be used to discover basic common features involved in neuropathic pain.

Further animal models for pain are considered in an article of Walker et al. 1999 Molecular Medicine Today 5:319-321, comparing models for different types of pain, which are acute pain, chronic/inflammatory pain and chronic/neuropathic pain, on the basis of behavioral signs.

Object of the present invention is to provide a target for examination and development of a medicament for the treatment of pain.

### SUMMARY OF THE INVENTION

This object is met by a method for the development of a medicament for the treatment of pain comprising the use of a polynucleotide sequence encoding Cathepsin Y or homologues or fragments thereof or the according polypeptide.

In on preferred embodiment of the present invention a method is disclosed for identifying potential therapeutic agents for treating pain. The method comprises: a) providing a test cell capable of expressing a Cathepsin Y gene or homologues or fragments thereof; b) contacting said test cell with the potential therapeutic agent; c) detecting a level of expression of the Cathepsin Y gene in said test cell; d) comparing the level of expression of the Cathepsin Y gene in the test cell to a level of expression of the Cathepsin Y gene in a reference cell whose disease stage is known; and e) identifying a difference in the expression levels of the Cathepsin Y gene in the test cell and reference cell, thereby identifying the potential therapeutic agent for treating pain.

In a variation to the above method, the expression of the Cathepsin Y gene is determined by at least one method selected from the group consisting of PCR of a cDNA, hybridizing a sample DNA, and detecting a Cathepsin Y protein.

In a preferred variation of the method, the pain is neuropathic pain.

In another preferred embodiment of the present invention a method is disclosed for identifying a therapeutic agent for treating pain, comprising: a) incubating a sample comprising a Cathepsin Y protein, a test compound/agent, and a polypeptide which is a target of the Cathepsin Y protein for proteolysis; b) determining an aminoterminal amino acid of a peptide resulting from the proteolysis of said target polypeptide or the amount of free amino acids in the sample after step (a); and c) comparing the aminoterminal amino acid of the peptide or the amount of free amino acids with a result obtained in a sample which does not contain the test compound/agent, thereby identifying the therapeutic agent for treating pain.

Preferably this method is also directed to identifying agents for treating pain which is neuropathic pain.

In another aspect, the present invention is directed at a pharmaceutical composition for the treatment of pain, comprising a compound having a general formula:

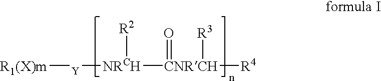

wherein:

R is selected from the group consisting of hydrogen, alkyl of from 1 to 6 carbon atoms, and where R and R2 are joined to form a ring structure of from 4 to 10 carbon atoms,

R′ is selected from the group consisting hydrogen, alkyl of from 1 to 6 carbon atoms, and where R′ and R³ are joined to form a ring structure of from 4 to 10 carbon atoms,

R₁ is selected from the group consisting of alkyl of from 1 to 4 carbon atoms substituted with from 1 to 5 substituents selected from the group consisting of (a) aryl of from 6 to 10 carbon atoms, (b) aryl of from 6 to 10 carbon atoms substituted with 1 to 3 substituents selected from the group consisting of alkyl of from 1 to 6 carbon atoms, aryl of from 6 to 10 carbon atoms, alkoxy of from 1 to 6 carbon atoms, aryloxy of from 6 to 10 carbon atoms, hydroxy, cyano, halo and amino, (c) cycloalkyl of from 3 to 8 carbon atoms and (d) heterocycles of from 3 to 14 carbon atoms having from 1 to 3 heteroatoms selected from the group consisting of nitrogen, oxygen and sulfur wherein said substituted alkyl group is optionally further substituted with from 1 to 2 hydroxyl groups, alkenyl of from 2 to 4 carbon atoms substituted with from 1 to 4 substituents selected from the group consisting of (a) aryl of from 6 to 10 carbon atoms, (b) aryl of from 6 to 10 carbon atoms substituted with 1 to 3 substituents selected from the group consisting of alkyl of from 1 to 6 carbon atoms, aryl of from 6 to 10 carbon atoms, alkoxy of from 1 to 6 carbon atoms, aryloxy of from 6 to 10 carbon atoms, hydroxy, cyano, halo and amino, (c) cycloalkyl of from 3 to 8 carbon atoms and (d) heterocycles of from 3 to 14 carbon atoms having from 1 to 3 heteroatoms selected from the group consisting of nitrogen, oxygen and sulfur, aryl of from 6 to 10 carbon atoms, aryl of from 6 to 10 carbon atoms substituted with 1 to 3 substituents selected from the group consisting of alkyl of from 1 to 6 carbon atoms, aryl of from 6 to 10 carbon atoms, alkoxy of from 1 to 6 carbon atoms, aryloxy of from 6 to 10 carbon atoms, hydroxy, cyano, halo and amino, fluorenyl, heterocycles of from 3 to 14 carbon atoms having from 1 to 3 heteroatoms selected from the group consisting of nitrogen, oxygen and sulfur;

R² and R³ are independently D- or L-amino acid side chains of at least 2 carbon atoms with the proviso that said amino acid side chains do not include the proline side chain;

R⁴ is selected from the group consisting of —C(O)CH═N═N, —CH₂OH, —C═NOH, and —C(O)R⁵, where R⁵ is hydrogen, alkyl of from 1 to 6 carbon atoms, haloalkyl of from 1 to 6 carbon atoms and 1 to 2 halo groups, alkoxy of from 1 to 6 carbon atoms, —NR⁶R⁷ where R⁶ and R⁷ are independently selected from the group consisting of hydrogen and alkyl of from 1 to 6 carbon atoms, and aryl of from 6 to 10 carbon atoms, and —N(CH₃)OCH₃;

X is selected from the group consisting of —O—, —NR⁹—, and —S— where R⁹ is selected from the group consisting of hydrogen, alkyl of from 1 to 6 carbon atoms and aryl of from 6 to 10 carbon atoms;

Y is selected from the group consisting of —C(O)— and —C(S)—;

m is equal to zero or one; and

n is equal to zero, one or two, or pharmaceutically acceptable salts thereof with the proviso that when R¹ is 1-naphthyl, R² is —CH(CH₃)₂ (L-isomer), R³ is —CH₂—Ø (L-isomer), Y is —C(O)—, m is zero and n is one, then R⁴ is not —N(CH₃)OCH₃, with the further proviso that when R′ is diphenylmethyl, R² is p-(benzyloxy)benzyl (L-isomer), Y is —C(O)—, and m and n are zero, then R⁴ is not —N(CH₃)OCH₃, and with still the further proviso that when R¹ is (1,2diphenyl)ethenyl, Y is —C(O)—, R² is —CH₂-Ø (L-isomer), and m and n are zero, then R⁴ is not —N(CH₃)OCH₃;

wherein said compound downregulates Cathepsin Y activity.

These compositions are preferably active in treating pain which is neuropathic pain.

In a preferred variation to the pharmaceutical composition of the present invention, the compound is selected from the group consisting of:

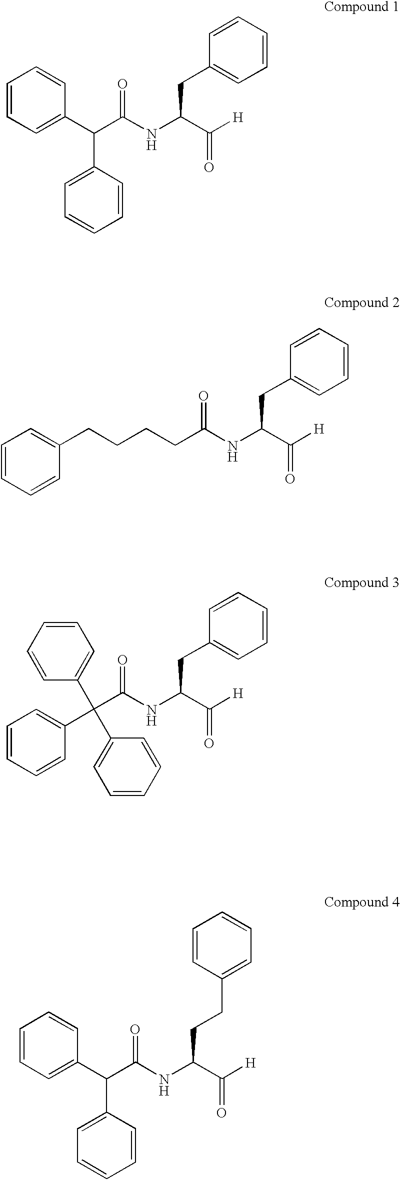

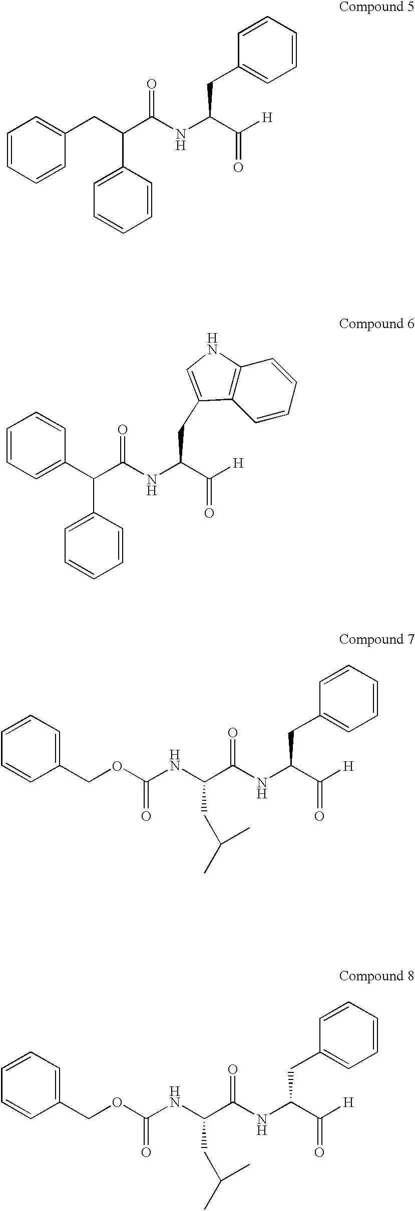

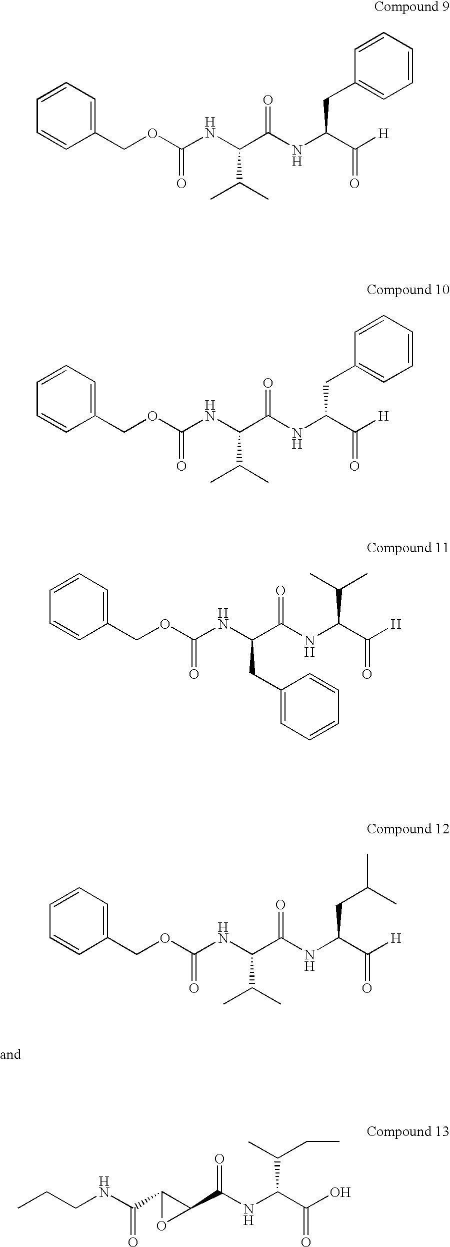

Another pharmaceutical composition for the treatment of pain disclosed in accordance with the present invention, comprising a nucleic acid sequence which is an “antisense” sequence compared to a nucleic acid sequence encoding Cathepsin Y of SEQ ID NO: 2, or SEQ ID NO 4, or homologues or fragments thereof. This composition is also directed at treating neuropathic pain.

### BRIEF DESCRIPTION OF THE DRAWINGS

FIG. 1 shows comparison experiments of three pain models: chronic constriction injury (CCI), tight ligation of the partial sciatic nerve (PSL) and tight ligation of spinal nerves (SNL). Expression levels of Cathepsin Y gene are compared over a time period of 28 days to sham operated controls. Cathepsin Y is described as differentially expressed when the sequence is up or down regulated at one time point in at least two of the three models. Over time the expression pattern can be determined as up-regulated in one to four time points; as down regulated in one to four time points or as mixed regulated if the type of regulation changes between up and down regulation at different time points.

X-axis describes the four time points analyzed by DEPD, 1=day one post operation, 2=day 7 post operation, 3=day 14 post operation, 4=day 28 post operation. The Y-axis shows Δh which represents the normalized difference of expression (peak height) of a certain transcript between a control group and a treated group. x-fold difference in gene expression is calculated by

$$\frac{1 + Δ h}{1 - Δ h}$$

0=no change to control, +=up regulation, −=down regulation; (0.2 =1.5 fold; 0.3=1.86 fold; 0.4=2.33 fold; 0.5=3 fold).

### DETAILED DESCRIPTION OF THE PREFERRED EMBODIMENT

According to the invention it has been found that Cathepsin Y is differentially expressed under pain, particularly under neuropathic pain. In the present invention it is shown that in three different rat models Cathepsin Y expression is upregulated under neuropathic pain. Examinations have been carried out in the models of Bennett, Seltzer and Kim & Chung (Bennett & Xie 1988 Pain 33:87-107, Seltzer et al. 1990 Pain 43:205-218 and Kim & Chung 1992 Pain 50:355-363).

In the following the models are designated as follows:

The model of Bennett is based on the chronic constriction injury by the loose ligation of the sciatic nerve. Therefore this model is designated as “CCI model”.

The model of Seltzer et al. is based on the tight ligation of the partial sciatic nerve and is therefore designated as “PSL model”.

The model of Kim & Chung is based on the tight ligation of spinal nerves and is therefore designated as “SNL model”.

These designations correspond to the designations used in the cited literature.

The three models are explained more in detail in the literature and in the examples below.

The term “polynucleotide sequence” or “nucleic acid sequence” designates in the present application any DNA or RNA sequence, independent of the length. Thus this term can describe short sequences like PCR primers or probes for hybridization, as well as whole genes or cDNA of these genes.

The term “polypeptide” or “amino acid sequence” designates a chain of amino acids, independent from their length, but in any case more than one amino acid.

As “homologues” of polynucleotide sequences such polynucleotide sequences are designated which encode the same type of protein as the polynucleotide sequence described herein, particularly a homologous polynucleotide sequence that encodes a polypeptide with the same type of activity. Accordingly as “homologues” of a polypeptide polypeptides are designated which have an amino acid sequence, wherein at least 70%, preferably 80%, more preferably 90% of the amino acids are identical to the protein of the present invention and wherein the replaced amino acids preferably are replaced by homologous amino acids. As “homologous” amino acids are designated which have similar features concerning hydrophobicity, charge, steric features, etc. Most preferred are amino acid sequences, containing the species- or family-dependent differences of the amino acid sequence. Particularly as “homologues” sequences are designated those which correspond to one of the cited sequences in another species or individuum. For example, if in the present invention a rat model is used and the cited polynucleotide sequence encodes the rat protein, the according polynucleotide sequence and protein of a mouse in a mouse model is designated as “homologue”. Further, splice variants and members of gene families are designated as homologues.

“Fragments” of a polynucleotide sequence are all polynucleotide sequences which have at least 10 identical base pairs compared to the polynucleotide sequence shown in the present application or by the gene represented by these polynucleotide sequence. The term “fragment” encloses therefore such fragments as primers for PCR, probes for hybridization, DNA fragments included in DNA vectors like plasmids, cosmids BACs or viral constructs, as well as shortened splice variants of the genes identified herein. As a fragment of a protein (polypeptide) amino acid sequences are designated which have at least three amino acids, preferably at least 10 amino acids. Therefore fragments serving as antigens or epitopes are enclosed in this designation.

In the present application the term “sequence” is used when either a polynucleotide sequence (=nucleic acid sequence) or a polypeptide (=amino acid sequence) or a protein is meant. That means when it is irrelevant which type of sequence is used the type is not designated particularly, but with the more common term “sequence”.

The basis of the methods and assays described in the present application is the examination of Cathepsin Y which is differentially expressed under pain or during development of pain. For the examination necessary for the development of any medicament each sequence can be used which allows the determination of the expression rate of the Cathepsin Y gene or the activity of the polypeptide/protein. The considered sequence can be the polynucleotide sequences SEQ ID NO. 1, SEQ ID NO. 2 or SEQ ID NO. 4, respectively or homologues or fragments thereof, as well as the polypeptides encoded thereby (SEQ ID NO. 3 or SEQ ID NO. 5, respectively) or homologues or fragments thereof.

According to the invention it has been found out, that the Cathepsin Y gene represented by the polynucleotide sequence of SEQ ID No: 2 is differentially expressed correspondingly in three different models for pain, which are the above described models “CCI”, “PSL”, “SNL”.

Therefore the present invention provides a target for the development of medicaments for the treatment of pain, particularly for the treatment of neuropathic pain. Cathepsin Y has not yet been regarded in relation to pain.

JP2000157263 describes a human Cathepsin Y useful in the elucidation and therapy for Alzheimer's disease, neurodegenerative diseases, cancers and the like. Comparison of the nucleic acid sequence shown in this publication with the sequences of common databases, however, shows that the sequence provided in JP2000157263 corresponds to Cathepsin F, not to Cathepsin Y.

WO 96/39194 teaches that the Cathepsin Y protein is involved in the secretion of β-amyloid peptide (β-AP) from cells, resulting in the development of Alzheimer's disease. The nucleic acid sequence as well as the protein sequence of Cathepsin Y is provided. For the treatment of Alzheimer's disease the inhibition of this protein is proposed, therefore inhibitors of Cathepsin Y are examined.

According to the present invention the Cathepsin Y sequences (polynucleotide sequence or polypeptide) can be used in any test system which allows the determination of either the expression rate of the gene or of the activity of the polynucleotide sequence or of the polypeptide/protein.

For determination and comparison of the expression levels of at least one of the genes identified in the present invention any of the commonly known methods can be used, either on RNA/cDNA level or on protein level. For example PCR, hybridization, microarray-based methods, Western blot or 2-D protein gel analysis are suitable methods. One preferred method is the digital expression pattern display method (DEPD method), explained in detail in WO99/42610. The method used for determination of expression levels is not restrictive, as long as expressed amounts can be quantified.

Activity of the Cathepsin Y protein can be determined by contacting the protein with polypeptides under acid pH conditions and determining the concentration of free amino acids after cleavage. Activity of the Cathepsin Y protein is a carboxypeptidase activity. Methods for determination of Cathepsin Y activity are explained in detail in international patent application WO 96/39194.

Expression and activity of Cathepsin Y can further be determined in assay systems or models. Such assay systems may be in vivo, ex vivo or in vitro assays, for example a cellular assay system comprising cells expressing Cathepsin Y, or an assay comprising isolated Cathepsin Y.

In any assay or model at least one of the sequences is contacted with the compound(s) to be tested and samples are obtained, wherein expression levels or activity of the sequences are determined and compared to non-treatment conditions.

For examination of the expression rate of the gene, animal models can be used. As such a model any animal can be used wherein the necessary preparations can be carried out, however, mammalian models are preferred. Even more preferred are rodents and lagomorphae, particularly preferred are rats, mice and rabbits. The most preferred animal model of the present invention is a rat model.

Dependent from the model used, the samples can be derived from whole blood, cerebrospinal fluid (CSF) or whole tissue, from cell populations isolated from tissue, cerebrospinal fluid (CSF) or blood or from single cell populations (i.e. cell lines).

In one embodiment of the invention cellular assays can be used. Preferred cells for cellular assays are eukaryotic cells, more preferably mammalian cells. Most preferred are neuronal-like cells, like SHSY5Y (neuroblastoma cell line) or COS cells (African green monkey, kidney cells); CHO cells (Chinese hamster ovary), HEK-293 cells (human embryonic kidney).

The Cathepsin Y sequences of the present invention can be used for diagnosing a pain status, particularly a neuropathic pain status of a human outside of the living body by determining the expression levels or activity of Cathepsin Y sequences in comparison to the non-disease status. During treatment period of a patient the expression or activity of the sequences can also be used for assessing the efficacy of pain treatment outside of the body. In these cases blood, CSF or tissue is removed from the patient and expression or activity is determined in the samples.

The disease which is considered in the present invention is pain. The preferred types of pain are persistent/central pain, inflammatory pain (acute or chronic) or neuropathic pain, particularly chronic neuropathic pain. The most preferred type is neuropathic pain.

Examples of such diseases are diabetic neuropathy, post-herpetic neuralgia, trigeminal neuralgia, cancer associated pain, spinal cord injury, multiple sclerosis, phantom pain, post-stroke pain, HIV associated pain, low back pain associated neuropathic pain, complex regional pain syndromes, like reflex sympathetic dystrophy and causalgia, myofacial syndromes or idiopathic pain conditions.

Independent whether the efficacy of pain treatment or the efficiency of a compound for the treatment of pain shall be examined or a pain status is diagnosed or characterized, determination of the expression level or the activity of the sequences is carried out outside of a living body.

As mentioned above, detection of the expression of the genes can be carried out by any method known in the art. The method of detection is not limiting the invention.

Expression levels can be detected either on the basis of a polynucleotide sequence or by detecting the according polypeptide, encoded by said polynucleotide sequence.

Preferred methods for detection and determination of the gene expression levels are PCR of cDNA, generated by reverse transcription of expressed mRNA, hybridization of polynucleotides (Northern-, Southern Blot systems, in situ hybridization), DNA microarray-based technologies, detection of the according peptides or protein via, e.g., Western Blot systems, 2-dimensional gel analysis, protein micro-array based technologies or quantitative assays like e.g. ELISA tests.

The most preferred method for quantitative analysis of the expression levels is the differential expression pattern display method (DEPD), described in detail in WO99/42610.

By using any of the Cathepsin Y sequences (polynucleotide sequence or polypeptide/protein) the efficiency of compounds for reducing the expression or the activity of the polynucleotide sequence or the protein can be tested. Therefore the sequences of the present invention can be used for identifying therapeutic agents and their efficiency for the treatment of pain.

For example a method can be used comprising:

a) providing a test cell population comprising cells capable of expressing the Cathepsin Y gene(s) or homologues or fragments thereof;

b) contacting said test cell population with the test therapeutic agent;

c) detecting the expression of the Cathepsin Y gene(s) in said test cell population;

d) comparing the expression of the gene(s) in the test cell population to the expression of the gene(s) in a reference cell population whose disease stage is known; and

e) identifying a difference in expression levels of the considered sequences, if present, in the test cell population and the reference cell population, thereby identifying a therapeutic agent for treating pain.

For this method an animal model can be used or test cells can be obtained from a subject, an animal model or cell cultures of fresh cells or cell lines. Further in vitro assays may be used.

Agents or compounds which have any influence on the expression rate of the Cathepsin Y gene(s) might be used as a medicament for the treatment of pain. A method examining the expression rate of the Cathepsin Y gene therefore can be used for testing agents and compounds for their efficiency for treatment of pain. Which model is used is not relevant, as long as the model allows to determine differences in expression amounts.

In such a model cells are contacted with the interesting agent or compound and expression of Cathepsin Y gene is determined in comparison to the expression in cells which never have been contacted to the according agent/compound. Contacting the cells either can be effected by administering the agent/compound to an animal or by contacting isolated cells of tissue or blood or cells of cell lines in culture with the agent/compound.

By examination of the influence the considered agent/compound has to the expression of the Cathepsin Y gene the efficacy of the agent/compound for treating pain can be estimated. This allows the decision whether it is worthwhile to develop a medicament containing such an agent or compound for the treatment of pain.

Whether the expression is determined on the basis of mRNA generation (transcription level) or on basis of protein generation (translation level) is not relevant, as long as the difference of the expression rate can be determined. Therefore the polynucleotide sequence, as well as the polypeptide or protein shown in the present application can be used for the development of a medicament.

The development of a medicament can be desirable for example if the considered compound/agent have any influence on the regulation of the expression rate or on the activity of any polynucleotide sequence or polypeptide of the present invention. Particularly if the agent or compound decreases the expression rate or the activity of Cathepsin Y it might be a candidate for the development of a medicament.

Said influence of a compound or agent can be examined by a method comprising contacting a sample comprising the nucleic acid sequence or homologues or fragment thereof or the according polypeptides of the present invention with a compound that binds to said sequence in an amount sufficient to determine whether said compound modulates the expression rate or the activity of the polynucleotide or polypeptide sequence.

By such a method a compound or agent can be determined modulating the expression rate or activity of the nucleic acid sequence or homologues or fragments thereof or the according polypeptides.

A method for determination of the efficiency of a compound/agent for the treatment of pain by determination of the activity of the Cathepsin Y protein comprises, for example:

a) contacting the Cathepsin Y protein with a compound/agent in the presence of a known polypeptide which is a target of the Cathepsin Y;

b) determining the aminoterminal amino acid of the peptide or the amount of free amino acids in the sample after incubation;

c) comparing the aminoterminal amino acid of the peptide or the amount of free amino acids with the result in a sample which doesn't contain the compound/agent.

If a compound/agent is able to decrease or inhibit the activity of the Cathepsin Y protein it might be useful as a medicament for the treatment of pain.

Examples for compounds which can decrease Cathepsin Y activity are compounds of formula I:

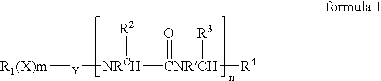

Wherein:

R is selected from the group consisting of hydrogen, alkyl of from 1 to 6 carbon atoms, and where R and R2 are joined to form a ring structure of from 4 to 10 carbon atoms,

R′ is selected from the group consisting hydrogen, alkyl of from 1 to 6 carbon atoms and where R′ and R³ are joined to form a ring structure of from 4 to 10 carbon atoms,

R¹ is selected from the group consisting of alkyl of from 1 to 4 carbon atoms substituted with from 1 to 5 substituents selected from the group consisting of (a) aryl of from 6 to 10 carbon atoms, (b) aryl of from 6 to 10 carbon atoms substituted with 1 to 3 substituents selected from the group consisting of alkyl of from 1 to 6 carbon atoms, aryl of from 6 to 10 carbon atoms, alkoxy of from 1 to 6 carbon atoms, aryloxy of from 6 to 10 carbon atoms, hydroxy, cyano, halo and amino, (c) cycloalkyl of from 3 to 8 carbon atoms and (d) heterocycles of from 3 to 14 carbon atoms having from 1 to 3 heteroatoms selected from the group consisting of nitrogen, oxygen and sulfur wherein said substituted alkyl group is optionally further substituted with from 1 to 2 hydroxyl groups, alkenyl of from 2 to 4 carbon atoms substituted with from 1 to 4 substituents selected from the group consisting of (a) aryl of from 6 to 10 carbon atoms, (b) aryl of from 6 to 10 carbon atoms substituted with 1 to 3 substituents selected from the group consisting of alkyl of from 1 to 6 carbon atoms, aryl of from 6 to 10 carbon atoms, alkoxy of from 1 to 6 carbon atoms, aryloxy of from 6 to 10 carbon atoms, hydroxy, cyano, halo and amino, (c) cycloalkyl of from 3 to 8 carbon atoms and (d) heterocycles of from 3 to 14 carbon atoms having from 1 to 3 heteroatoms selected from the group consisting of nitrogen, oxygen and sulfur, aryl of from 6 to 10 carbon atoms, aryl of from 6 to 10 carbon atoms substituted with 1 to 3 substituents selected from the group consisting of alkyl of from 1 to 6 carbon atoms, aryl of from 6 to 10 carbon atoms, alkoxy of from 1 to 6 carbon atoms, aryloxy of from 6 to 10 carbon atoms, hydroxy, cyano, halo and amino, fluorenyl, heterocycles of from 3 to 14 carbon atoms having from 1 to 3 heteroatoms selected from the group consisting of nitrogen, oxygen and sulfur;

R² and R³ are independently D- or L-amino acid side chains of at least 2 carbon atoms with the proviso that said amino acid side chains do not include the proline side chain;

R⁴ is selected from the group consisting of —C(O)CH═N═N, —CH₂OH, —C═NOH, and —C(O)R⁵, where R⁵ is hydrogen, alkyl of from 1 to 6 carbon atoms, haloalkyl of from 1 to 6 carbon atoms and 1 to 2 halo groups, alkoxy of from 1 to 6 carbon atoms, —NR⁶R⁷ where R⁶ and R⁷ are independently selected from the group consisting of hydrogen and alkyl of from 1 to 6 carbon atoms, and aryl of from 6 to 10 carbon atoms, and —N(CH₃)OCH₃;

X is selected from the group consisting of —O—, —NR⁹—, and —S—, where R⁹ is selected from the group consisting of hydrogen, alkyl of from 1 to 6 carbon atoms and aryl of from 6 to 10 carbon atoms; Y is selected from the group consisting of —C(O)— and —C(S)—;

m is equal to zero or one; and

n is equal to zero, one or two,

or pharmaceutically acceptable salts thereof,

with the proviso that when R¹ is 1-naphthyl, R² is —CH(CH₃)₂ (L-isomer), R³ is —CH₂—Ø (L-isomer), Y is —C(O)—, m is zero and n is one, then R⁴ is not —N(CH₃)OCH₃,

with the further proviso that when R′ is diphenylmethyl, R² is p-(benzyloxy)benzyl (L-isomer), Y is —C(O)—, and m and n are zero, then R⁴ is not —N(CH₃)OCH₃, and

with still the further proviso that when R¹ is (1,2diphenyl)ethenyl, Y is —C(O)—, R² is —CH₂-Ø (L-isomer), and m and n are zero, then R⁴ is not —N(CH₃)OCH₃.

In a preferred embodiment:

R¹ includes benzyl, trityl, diphenylmethyl, 4-phenylbutyl, 2-phenylethyl, naphthyl, pyridyl, fluorenyl, xanthanilyl, and the like;

R² and R³ are independently side chains of a D- or L- amino acid having at least 2 carbon atoms with the proviso that R² and R³ are not proline. Such side chains refer to the R⁸ substituent found on naturally occurring and synthetic amino acids of the formula H₂NCHR⁸COOH. Side chains of naturally occurring amino acids include, by way of example only, those where R⁸ is the L-isomer of (CH₃)₂CH— (valine), (CH₃)₂CHCH₂ (leucine), CH₃CH₂CH(CH₃)— (isoleucine), ØCH₂— (phenylalanine), (3-indolyl)—CH₂— (tryptophan), CH₃SCH₂CH₂— (methionine), CH₃CH(OH) (threonine), p—HO-Ø-CH₂— (tyrosine), H₂NC(O)CH₂— (asparagine), H₂NC(O)CH₂CH₂— (glutamine), HOC(O)CH₂— (aspartic acid), HOC(O)CH₂CH₂— (glutamic acid), H₂NCH₂CH₂CH₂CH₂— (lysine), H₂NC(NH)NHCH₂CH₂CH₂— (arginine), 4-imidazolyl-CH₂— (histidine) and the like.

Side chains of synthetic amino acids include the D-isomer of the above noted naturally occurring amino acids as well as those where R⁸ is selected from the group consisting of alkyl of from 2 to 6 carbon atoms (where the alkyl group does not occur in naturally occurring amino acids), cycloalkyl of from 3 to 8 carbon atoms, and alkyl of from 1 to 6 carbon atoms substituted with from 1 to 2 substituents selected from the group consisting of:

aryl of from 6 to 10 carbon atoms,

aryl of from 6 to 10 carbon atoms substituted with from 1 to 3 substituents selected from the group consisting of alkyl of from 1 to 6 carbon atoms,

alkoxy of from 1 to 6 carbon atoms, aryl of from 6 to 10 carbon atoms, and

aryloxy of from 6 to 10 carbon atoms, and

heteroaryl of from 3 to 14 carbon atoms having from 1 to 3 heteroatoms selected from the group consisting of nitrogen, oxygen and sulfur (where the substituted alkyl group does not occur in naturally occurring amino acids).

Particularly preferred amino acid side chains include the D- and L-isomers of valine, leucine, phenylalanine, tryptophan and isoleucine.

R⁴ preferably is —CH═N═N or—C(O)H.

X preferably is —O—.

Y preferably is —C(O)—.

m and n preferably are 0 or 1.

In the present application -Ø means a phenyl residue.

Preferred compounds include, by way of example, the following compounds as defined by formula II below, including all isomers thereof, wherein the amino acid side chain for R² and R³ is indicated beneath the R² and R³ substituent:

| | |
|---|---|
| 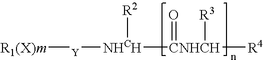 | formula II |

| R | ′X | m | Y | R² | n | R³ | R⁴ |
|---|---|---|---|---|---|---|---|
| Ø-CH₂— | 0 | 1 | —C(O)— | —CH(CH₃)₂ | 1 | —CH₂-Ø | —C(O)CH═N═N |
| | | | | (valine) | | (phenylalanine) | |
| Ø-CH₂— | 0 | 1 | —C(O)— | —CH₂-Ø | 1 | —CH₂-Ø | —C(O)CH═N═N |
| | | | | (phenylalanine) | | (phenylalanine) | |
| (Ø)_{2-CH—} | — | 0 | —C(O)— | —CH₂-Ø | 0 | — | —C(O)H |
| | | | | (phenylalanine) | | | |
| Ø-(CH₂)₄— | — | 0 | —C(O)— | —CH₂-Ø | 0 | — | —C(O)H |
| | | | | (phenylalanine) | | | |
| (Ø)_{3-C—} | — | 0 | —C(O)— | —CH₂-Ø | 0 | — | —C(O)H |
| | | | | (phenylalanine) | | | |
| (Ø)_{2-CH—} | — | 0 | —C(O)— | —CH₂—CH₂-Ø | 0 | — | —C(O)H |
| | | | | (homophenylalanine) | | | |
| ØCH═C(Ø) | — | 0 | —C(O)— | —CH₂-Ø | 0 | — | —C(O)H |
| | | | | (phenylalanine) | | | |
| (Ø)_{2-CH—} | — | 0 | —C(O)— | —CH₂-(3-indolyl) | 0 | — | —C(O)H |
| Ø-CH₂— | 0 | 1 | —C(O)— | —CH₂-Ø | 1 | —CH(CH₃)₂ | —C(O)H |
| | | | | (phenylalanine) | | (valine) | |
| Ø-CH₂— | 0 | 1 | —C(O)— | —CH(CH₃)₂ | 1 | —CH₂—CH(CH₃)₂ | —C(O)H |
| | | | | (valine) | | (leucine) | |

The term “heterocycles containing from 3 to 14 carbon atoms and 1 to 3 heteroatoms selected from the group consisting of nitrogen, oxygen and sulfur” refers to saturated and unsaturated heterocyclic groups having the requisite number of carbon atoms and heteroatoms. Suitable heterocyclic groups include, by way of example, furazanyl, furyl, imidazolidinyl, imidazolyl, imidazolinyl, indolyl, isothiazolyl, isoxazolyl, morpholinyl (e.g., morpholino), oxazolyl, piperazinyl (e.g., 1-piperazinyl), piperidyl (e.g., 1-piperidyl, piperidino), pyranyl, pyrazinyl, pyrazolidinyl, pyrazolinyl, pyrazolyl, pyridazinyl, pyridyl, pyrimidinyl, pyrrolidinyl (e.g., 1-pyrrolidinyl), pyrrolinyl, pyrrolyl, quinoxalinyl, thiadiazolyl, thiazolyl, thienyl, thiomorpholinyl (e.g., thiomorpholino), triazolyl, and xanthanilyl.

If in the present application a chemical group is described as containing for example “from 1 to 6 carbon atoms” or “from 6 to 10 carbon atoms” or “from 1 to 4 heteroatoms” it is meant that the group can contain either 1 or 2 or 3 or 4 or 5 or 6, or 6 or 7 or 8 or 9 or 10, or 1 or 2 or 3 or 4 of said atoms, respectively.

Heterocyclic groups can be substituted or unsubstituted. Where the heterocyclic group is substituted, the substituents are selected from alkyl of from 1 to 6 carbon atoms, alkoxy of from 1 to 6 carbon atoms, aryl of from 6 to 10 carbon atoms, aryloxy of from 6 to 10 carbon atoms, and halo.

Preferred heterocycles include well-known cyclic aromatic groups containing heteroatoms within the cyclic structure. Such groups include, by way of example, furyl, imidazolyl, oxazolyl, pyrazolyl, pyridyl, pyrimidinyl, thiazolyl, and triazolyl.

The term “alkyl” refers to straight and branched chain alkyl groups such as methyl, ethyl, n-propyl, iso-propyl, n-butyl, tert-butyl, sec-butyl, iso-butyl,n-pentyl, n-hexyl, 2-methylpentyl, and the like; whereas the term “alkoxy” refers to —O-alkyl substituents.

The term “aryl” refers to aromatic substituents comprising carbon and hydrogen such as phenyl, naphthyl and the like whereas the term aryloxy refers to —O-aryl substituents where aryl is as defined above.

The term “halo” or “halogen” refers to fluorine, chlorine, bromine and iodine and preferably fluorine and chlorine.

The term “pharmaceutically acceptable salts” refers to the non-toxic alkali metal, alkaline earth metal, and ammonium salts commonly used in the pharmaceutical industry including the sodium, potassium, lithium, calcium, magnesium, barium, ammonium, and protamine zinc salts, which are prepared by methods well known in the art. The term also includes non-toxic acid addition salts, which are generally prepared by reacting the compounds of this invention with a suitable organic or inorganic acid.

Representative salts include the hydrochloride, hydrobromide, sulfate, bisulfate, acetate, oxalate, valerate, oleate, laurate, borate, benzoate, lactate, phosphate, tosylate, citrate, maleate, fumarate, succinate, tartrate, and napsylate salts, and the like. The particular salt employed is not critical.

Particularly preferred embodiments of the compounds usable as a medicament for the treatment of pain by inhibiting protein activity of Cathepsin Y are the compounds 1 to 13 as shown in the following:

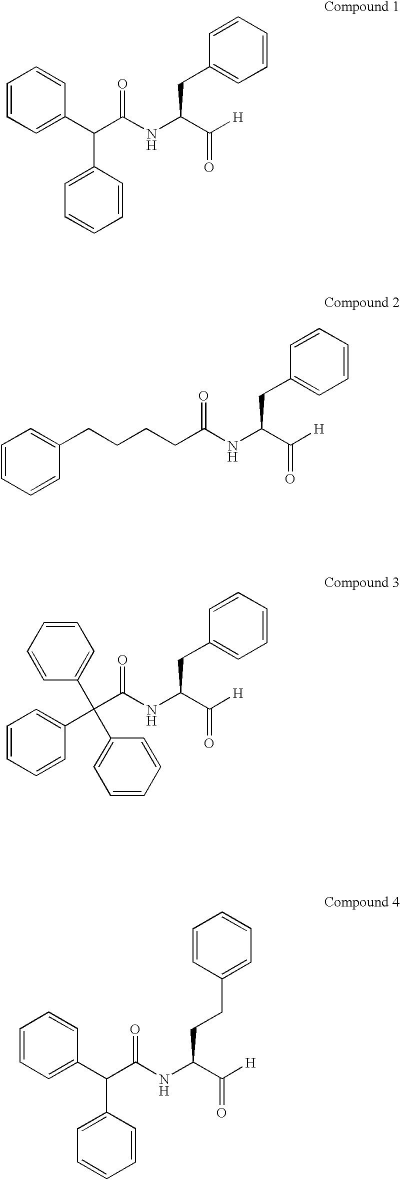

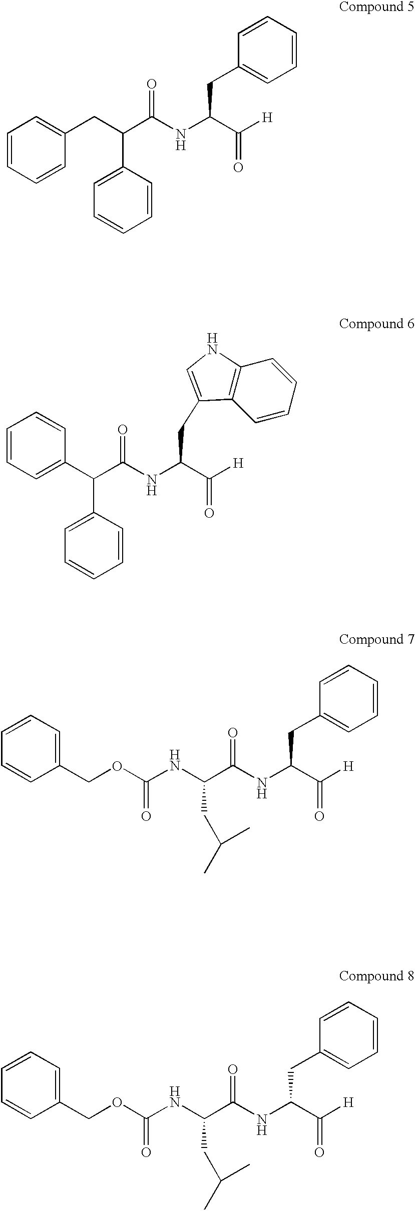

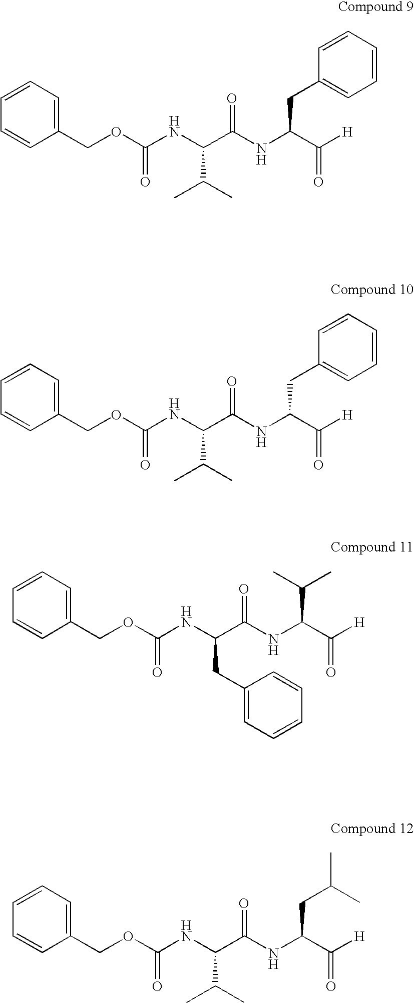

Preparation of the compounds 1 to 12 of the present application is explained in detail in WO96/39194.

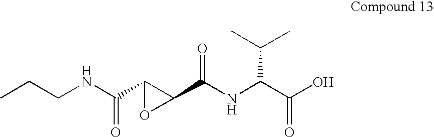

Preparation of compound 13 is explained in detail in the article of Therrien, C. et al. 2001 Biochemistry 40:2702-2711.

Furthermore the sequences of the present invention itself can be used as a medicament.

An example for such a use is the use of a polynucleotide sequence as an antisense agent. Antisense agents can hybridize to DNA or mRNA, inhibiting or decreasing transcription or translation, respectively. Thus, polynucleotide sequences of a gene which is increased in expression rate under pain can be used as antisense agents to decrease the expression rates of said gene. Further such polynucleotide sequences can be used for gene therapy.

A pharmaceutical composition comprising a polynucleotide sequence according to the present invention can be any composition which can serve as a pharmaceutical one. Salts or aids for stabilizing the sequence in the composition preferably are present.

For the determination of the expression of the relevant genes the generated sequences have to be detected. Therefore several reagents can be used, which are for example specific radioactive or non-radioactive (e.g., biotinylated or fluorescent) probes to detect nucleic acid sequences by hybridization, primer sets for the detection of one or several of the nucleic acid sequences by PCR, DNA microarrays, antibodies against one of the polypeptides, or epitopes, or antibody, or protein microarrays. Such reagents can be combined in a kit, which can be sold for carrying out any of the described methods.

For further examinations or experiments it might be desirable to include the nucleic acid sequence of the present invention into a vector or a host cell. By including the sequences in a host cell for example cellular assays can be developed, wherein the genes, polynucleotide sequences and the according proteins/polypeptides further can be used or examined.

Further the sequences defined in the present invention can be used to “design” new transgenic animals as models for pain. Therefore the animals are “created” by manipulating the gene(s) encoding Cathepsin Y in a way that their expression in the transgenic animal differs from the expression of the same gene(s) in the wild type. “Manipulation” preferably results in a different expression level of the Cathepsin Y in the transgenic animal compared to the wild type, or in a protein with different enzymatic activity. Whether the expression level or the protein activity becomes higher or lower than the wild type under the same conditions depends from the desired model for pain. Methods of gene manipulation and methods for the preparation of transgenic animals are commonly known to those skilled in the art.

The following examples are provided for illustration and are not intended to limit the invention to the specific example provided. EXAMPLE 1

### Preparation of Rat Models

A) The CCI Model: (Bennett & Xie 1988 Pain 3:87-107)

Nerve injury is created by loosely constrictive ligatures around rat sciatic nerve (4 ligatures). The ligatures evoke intraneural edema, the swelling is opposed by the ligatures of the nerve strangulates. The constrictions remain for at least a month (hence “chronic constriction injury”, CCI). It is known that the constriction injures nearly all of the nerves large myelinated axons; however, a variable but large percentage of the nerves unmyelinated axons remained intact. Although the nerve distal to the constriction is full of degeneration, the nerve proximal to the constriction appears normal, and there is no evidence of any primary afferent neuron dying. This model has a periphery that is innervated only by C-fibers and a greatly reduced number of A-delta fibers, and a spinal cord that is innervated by injured (but alive) A-beta low-threshold mechanoreceptors (AβLTMs), A-delta fibers (mostly injured, a few intact) and both injured and intact C-fiber afferents, many of which are nociceptors. This animal model provokes allodynia and hyperalgesia as well as spontaneous pain. Evidence of abnormal pain sensation is detected in the majority of CCI cases on the second PO day, and in nearly all cases by 5-7 days post injury. Abnormal pain sensation appears to reach peak severity in 10-14 days and to disappear in about 2 month when the pain is replaced by an apparently permanent state of hyperesthesia.

In detail, male Lewis rats (150-350 g body weight) were anesthetized with sodium pentobarbital (50 mg/kg i.p.). Thereafter the common sciatic nerve on the left side was exposed at the level of the middle of the thigh by blunt dissection through the biceps femoris. Proximal to the sciatic trifurcation, about 7 mm of nerve was freed from adhering tissues and 4 ligatures (4.0 chromic gut) were tied loosely around it with 1 mm spacing. The length of nerve affected was 4-5 mm long. The desired degree of constriction retarded, but did not arrest, circulation through the superficial epineurial vasculature and sometimes produced a small, brief twitch in the muscle surrounding exposure. Control group animals were sham ligated (only left site), which represents the same operative procedure as the ligated one but without nerve ligation.

Four time points for gene expression profiling were chosen: 1 day, 7 days, 14 days, and 28 days post operation. Animals were tested for mechanical allodynia (von Frey test) one day before surgery and within 30-60 min before tissue preparation (dorsal root ganglia, spinal cord and thalamus). Tissues were frozen on liquid nitrogen prior to RNA preparation.

B) The PSL Model (Seltzer, et al. 1990 Pain 43: 205-218)

Disorders in nocifensive behavior following noxious and non-noxious stimuli begin hours after partial sciatic nerve injury and last for at least 7 months. This model is characterized by complex combination of rapid onset, allodynia to touch, hyperalgesia, mirror image phenomena, and dependence on the sympathetic outflow. This model resembles therefore many of the symptoms described for causalgia in man. The rapid initiation of these disorders and their contralateral appearance suggest central reorganization. This model may serve as a model for sympathetically maintained pain (SMP).

In detail Lewis rats (male 150-350 g body weight) were anesthetized with sodium pentobarbital (50 mg/kg i.p.) followed by exposure of the left sciatic nerve at high-thigh level. Under 25× magnification, the dorsum of the nerve was carefully freed from surrounding tissues at a site near the trochanter just distal to the point at which the posterior biceps semitendinous (‘PBST’) nerve branches off the common sciatic nerve. An 8-0 silicon-treated silk suture was inserted into the nerve with a ⅜ curved, reversed-cutting mini-needle, and tightly ligated so that the dorsal ⅓ to ½ of the nerve thickness was trapped in the ligature.

Control group animals were sham ligated, which represents the same operative procedure as the ligated one but without nerve ligation.

Four time points for gene expression profiling were chosen: 1 day, 7 days, 14 days, and 28 days post operation. Animals were tested for mechanical allodynia (von Frey test) one day before surgery and 30-60 min before tissue preparation (dorsal root ganglia, spinal cord and thalamus). Tissues were frozen on liquid nitrogen prior to RNA preparation.

C) The SNL Model (Kim & Chung 1992 Pain 50:355-363)

In this model a tight ligation of the left L5 and L6 spinal nerves is leading to a long-lasting hyperalgesia to noxious heat, at least 5 weeks, of the affected foot. Long-lasting mechanical allodynia of the affected foot can be observed for at least 10 weeks. This model involves a complete ligation of spinal nerves L5 and L6 or only L5 (or L4), which is more reliable to other models where the number and types of ligated nerves are difficult to control.

In detail, male Lewis rats (150-350 g body weight) were anesthetized with sodium pentobarbital (50 mg/kg i.p.). Thereafter the sciatic spinal nerve ligation of the spinal nerve L5 was performed on the left site of the animals.

Control group animals were sham ligated (only left site), which represents the same operative procedure as the ligated ones but without nerve ligation.

Four time points for gene expression profiling were chosen: 1 day, 7 days, 14 days, and 28 days post operation. Animals were tested for mechanical allodynia (von Frey test) one day before surgery and 30-60 min before tissue preparation (dorsal root ganglia, spinal cord and thalamus). Tissues were frozen on liquid nitrogen prior to RNA preparation.

### EXAMPLE 2

### Determination of Expression Levels

Gene expression profiling by DEPD-analysis starts with the isolation of 5-10 μg total RNA. In a second step, double-stranded cDNA is synthesized. Through an enzymatic digest of the cDNA with three different type IIS restriction enzymes, three pools with short DNA-fragments containing single-stranded overhangs are generated. Afterwards, specific DNA-adaptor-molecules are ligated and in two subsequent steps 3.072 PCR reactions are performed by using 1024 different unlabelled 5′ primer and a common FAM-fluorescent labelled 3′-primer in the last PCR step. Subsequently, the 3072 PCR pools are analyzed on an automatic capillary electrophoresis sequencer.

Differential gene expression pattern of single fragments are determined by comparison of normalized chromatogram peaks from the control groups and corresponding operated animals.

### EXAMPLE 3

### Sequencing and Databank Analysis of the Obtained Sequences

Differentially expressed peaks are confirmed on polyacrylamide gels by using radioactive labelled 3′ primer instead of the FAM-fluorescent primer. Differentially expressed bands are cut from the gel. After a short elution step in 60 μl 10 mM Tris pH 8, fragments are re-amplified by PCR using the same primer as used in the DEPD analysis. Resulting PCR products are treated with a mixture of Exonuclease I and shrimp alkaline phosphatase prior to direct sequencing. Sequencing reactions are performed by using a sequencing kit like DYE-namic ET dye terminator sequencing kit, and subsequently analyzed by capillary electrophoresis (Megabace 1000, Amersham).

Prior to a BLAST analysis (Altschul et al. 1997 Nucleic Acids Res 25:3389-3402) against Genbank and Unigene, sequences are quality verified and redundant sequences or repetitive motifs are masked.

Results are shown in Table 1 and are compared to human sequence.

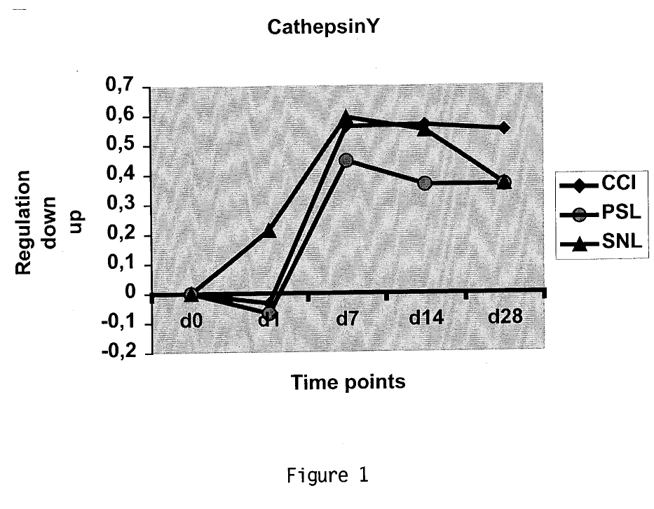

Expression of the Cathepsin Y gene over a time period of 28 days after preparation of the pain model is shown in FIG. 1.

| SEQ ID NO: | name | length | |
|---|---|---|---|
| 1 | EST of Cathepsin Y (rat) | 255 | bp |
| 2 | Rattus norvegicus Cathepsin Y DNA | 1387 | bp |
| 3 | rat Cathepsin Y protein | 306 | aa |
| 4 | human Cathepsin Y DNA | 1500 | bp |
| 5 | human Cathepsin Y protein | 303 | aa |

### EXAMPLE 4

### Enzymatic Activity of Cathepsin Y Protein

Cathepsin Y does not have the standard endopeptidic activity associated with proteases. In an effort to identify whether the enzyme has other proteolytic activity, a number of randomly selected synthetic oligopeptides were incubated with purified Cathepsin Y at pH 5.5 or 4.5 . Specifically, purified Cathepsin Y was incubated with the selected synthetic oligopeptides (50 μg/ml) at either pH 4.5 or pH 5.5, for 1 hour at 37° C. Samples were quenched by the addition of trifluoroacetic acid to 1% final concentration, then analyzed by reverse phase HPLC on a Vydac C18 column, using a gradient of increasing acetonitrile in 0.1% trifluoroacetic acid. Individual peaks (parent and new product(s)) were collected, then analyzed by acid hydrolysis followed by amino-acid analysis.

The results are summarized below:

| Parent Sequence | Product(s) |
|---|---|
| LFYDQSPTATI | LFYDQSPTAT |
| (SEQ ID NO: 6) | (aa 1-10 of the parent sequence) |
| | LFYDQSPTA |
| | (aa 1-9 of the parent sequence) |
| | LFYDQSPT |
| | (aa 1-8 of the parent sequence) |
| YKRDMVGGVVIA | YKRDMVGGVVI |
| (SEQ ID NO: 7) | (aa 1-11 of the parent sequence) |
| | YKRDMVGGVV |
| | (aa 1-10 of the parent sequence) |
| EGYYGNYGVYA | EGYYGNYGVY |
| (SEQ ID NO: 8) | (aa 1-10 of the parent sequence) |
| | EGYYGNYGV |
| | (aa 1-9 of the parent sequence) |
| FFDEPNPGVTIY | FFDEPNPGVT |
| (SEQ ID NO: 9) | (aa 1-10 of the parent sequence) |

The above peptides, as well as other peptides recited herein, are listed, per convention, from amino terminus to the carboxyl terminus.

The results from a number of such experiments (all not shown) suggest that the proteolytic activity of Cathepsin Y is a sequential removal of the carboxy terminal amino acids which is direct evidence for carboxypeptidase activity. No evidence of any endopeptidase or aminopeptidase activity was seen with any of these substrates. These data strongly suggest that the predominant proteolytic activity manifested by Cathepsin Y is carboxypeptidase activity.

The quantitative analysis of carboxypeptidase activity of Cathepsin Y is based on the detection of the new free amino-terminus generated on cleavage of a selected substrate. This is accomplished by reacting with the reagent ophthalaldehyde in an alkaline solution in the presence of 2-mercaptoethanol (Simons, et al. 1976 JACS 98:7098-7099). The peptide EGYYGNYGV was synthesized acetylated on its amino-terminus so that on cleavage by Cathepsin Y, the only free amino-terminus present in the reaction mixture will be that of the valine residue, cleaved off by the carboxypeptidase activity of the protease.

A standard curve was constructed by incubating varying concentrations of valine (0-20 μM) in 0.25 M sodium borate, pH 10, containing 0.05% 2-mercaptoethanol and 60 μg/ml o-phthalaldehyde, in individual wells of a 96 well microtiter plate. The resulting fluorescence is read in a plate reading Cytofluor (ex 340, em 460 nm). There is a linear increase in the signal proportional to the amount of free valine present

In order to determine the pH optimum of the enzyme, reaction mixtures were set up with enzyme and substrate (0.2 mg/ml) in a total reaction volume of 0.1 ml in individual wells of 96-well microtiter plates, in 20 mM sodium acetate buffers at different pH, with 0.1% 2-mercaptoethanol present. Control wells were set identically except for the presence of enzyme. At various timepoints after incubation at room temperature, the reactions were quenched by the addition of an equal volume of 0.45 M sodium borate, pH 10, with 0.25 mg/ml o-phthalaldehyde, and the fluorescence measured. In the absence of added enzyme, no measurable fluorescence is generated above background, even with extended incubations. In the presence of enzyme, there is a time-dependent increase in fluorescence. Based on this analysis, the pH optimum for carboxypeptidase activity was determined to be about 4.5, with the activity dropping off at both lower and higher pH.

Using this assay at pH 4.5, the ability of a number of compounds to inhibit the activity of Cathepsin Y was tested, by adding the desired concentration of the inhibitor in the incubation mixture along with enzyme and substrate, and measuring the decrease in fluorescence (if any) relative to enzyme control. Each of the compounds 1 to 13 tested in this analysis indicated inhibition of Cathepsin Y activity.

What is claimed is:

1. A method for identifying potential therapeutic agents for treating pain, comprising:
a) providing a test cell capable of expressing a Cathepsin Y gene or homologues or fragments thereof;
b) contacting said test cell with the potential therapeutic agent;
c) detecting a level of expression of the Cathepsin Y gene in said test cell;
d) comparing the level of expression of the Cathepsin Y gene in the test cell to a level of expression of the Cathepsin Y gene in a reference cell whose disease stage is known; and
e) identifying a difference in the expression levels of the Cathepsin Y gene in the test cell and reference cell, thereby identifying the potential therapeutic agent for treating pain.

2. The method of claim 1, wherein expression of the Cathepsin Y gene is determined by at least one method selected from the group consisting of PCR of a cDNA, hybridizing a sample DNA, and detecting a Cathepsin Y protein.

3. The method of claim 1, wherein said pain is neuropathic pain.

4. A method for identifying a therapeutic agent for treating pain, comprising:
a) incubating a sample comprising a Cathepsin Y protein, a test compound/agent, and a polypeptide which is a target of the Cathepsin Y protein for proteolysis;
b) determining an aminoterminal amino acid of a peptide resulting from the proteolysis of said target polypeptide or the amount of free amino acids in the sample after step (a);
c) comparing the aminoterminal amino acid of the peptide or the amount of free amino acids with a result obtained in a sample which does not contain the test compound/agent, thereby identifying the therapeutic agent for treating pain.

5. The method of claim 4, wherein said pain is neuropathic pain.

6. A pharmaceutical composition for the treatment of pain, comprising a compound having a general formula: 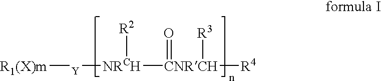
wherein:
R is selected from the group consisting of hydrogen, alkyl of from 1 to 6 carbon atoms, and where R and R2 are joined to form a ring structure of from 4 to 10 carbon atoms,
R′ is selected from the group consisting hydrogen, alkyl of from 1 to 6 carbon atoms, and where R′ and R³ are joined to form a ring structure of from 4 to 10 carbon atoms,
R₁ is selected from the group consisting of alkyl of from 1 to 4 carbon atoms substituted with from 1 to 5 substituents selected from the group consisting of (a) aryl of from 6 to 10 carbon atoms, (b) aryl of from 6 to 10 carbon atoms substituted with 1 to 3 substituents selected from the group consisting of alkyl of from 1 to 6 carbon atoms, aryl of from 6 to 10 carbon atoms, alkoxy of from 1 to 6 carbon atoms, aryloxy of from 6 to 10 carbon atoms, hydroxy, cyano, halo and amino, (c) cycloalkyl of from 3 to 8 carbon atoms and (d) heterocycles of from 3 to 14 carbon atoms having from 1 to 3 heteroatoms selected from the group consisting of nitrogen, oxygen and sulfur wherein said substituted alkyl group is optionally further substituted with from 1 to 2 hydroxyl groups, alkenyl of from 2 to 4 carbon atoms substituted with from 1 to 4 substituents selected from the group consisting of (a) aryl of from 6 to 10 carbon atoms, (b) aryl of from 6 to 10 carbon atoms substituted with 1 to 3 substituents selected from the group consisting of alkyl of from 1 to 6 carbon atoms, aryl of from 6 to 10 carbon atoms, alkoxy of from 1 to 6 carbon atoms, aryloxy of from 6 to 10 carbon atoms, hydroxy, cyano, halo and amino, (c) cycloalkyl of from 3 to 8 carbon atoms and (d) heterocycles of from 3 to 14 carbon atoms having from 1 to 3 heteroatoms selected from the group consisting of nitrogen, oxygen and sulfur, aryl of from 6 to 10 carbon atoms, aryl of from 6 to 10 carbon atoms substituted with 1 to 3 substituents selected from the group consisting of alkyl of from 1 to 6 carbon atoms, aryl of from 6 to 10 carbon atoms, alkoxy of from 1 to 6 carbon atoms, aryloxy of from 6 to 10 carbon atoms, hydroxy, cyano, halo and amino, fluorenyl, heterocycles of from 3 to 14 carbon atoms having from 1 to 3 heteroatoms selected from the group consisting of nitrogen, oxygen and sulfur;
R² and R³ are independently D- or L-amino acid side chains of at least 2 carbon atoms with the proviso that said amino acid side chains do not include the proline side chain;
R⁴ is selected from the group consisting of —C(O)CH═N═N, —CH₂OH, —C═NOH, and —C(O)R⁵, where R⁵ is hydrogen, alkyl of from 1 to 6 carbon atoms, haloalkyl of from 1 to 6 carbon atoms and 1 to 2 halo groups, alkoxy of from 1 to 6 carbon atoms, —NR⁶R⁷ where R⁶ and R⁷ are independently selected from the group consisting of hydrogen and alkyl of from 1 to 6 carbon atoms, and aryl of from 6 to 10 carbon atoms, and —N(CH₃)OCH₃;
X is selected from the group consisting of —O—, —NR⁹—, and —S— where R⁹ is selected from the group consisting of hydrogen, alkyl of from 1 to 6 carbon atoms and aryl of from 6 to 10 carbon atoms;
Y is selected from the group consisting of—C(O)— and —C(S)—;
m is equal to zero or one; and
n is equal to zero, one or two, or pharmaceutically acceptable salts thereof with the proviso that when R¹ is 1-naphthyl, R² is —CH(CH₃)₂ (L-isomer), R³ is —CH₂-Ø (L-isomer), Y is —C(O)—, m is zero and n is one, then R⁴ is not —N(CH₃)OCH₃, with the further proviso that when R′ is diphenylmethyl, R² is p-(benzyloxy)benzyl (L-isomer), Y is —C(O)—, and m and n are zero, then R⁴ is not —N(CH₃)OCH₃, and with still the further proviso that when R¹ is (1,2diphenyl)ethenyl, Y is —C(O)—, R² is —CH₂-Ø- (L-isomer), and m and n are zero, then R⁴ is not —N(CH₃)OCH₃;
wherein said compound downregulates Cathepsin Y activity.

7. The composition of claim 6, wherein said compound is selected from the group consisting of:

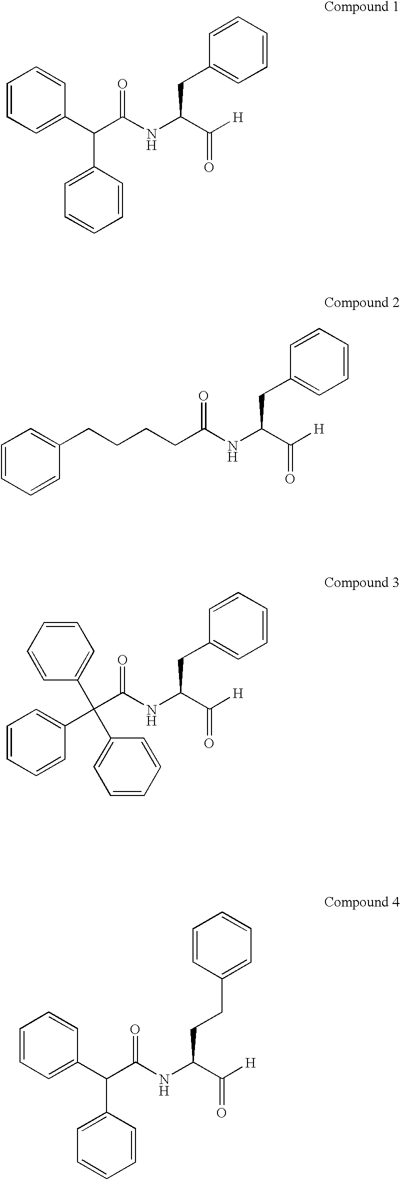

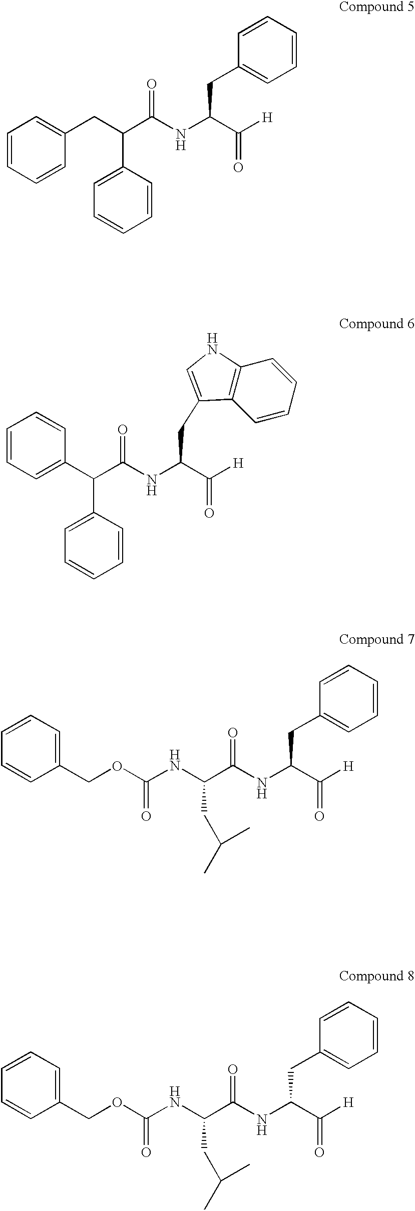

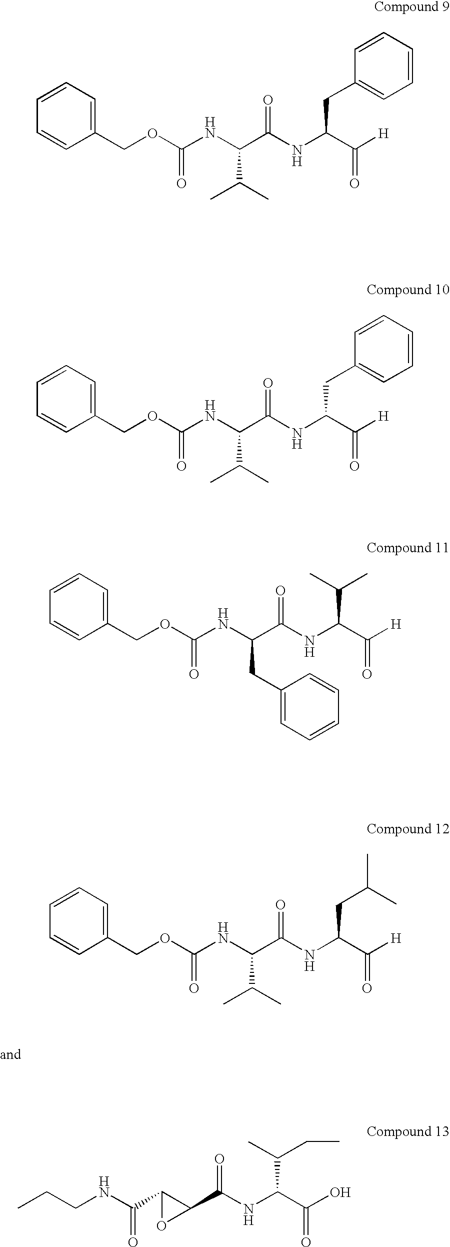

8. The method of claim 6, wherein said pain is neuropathic pain.

9. A pharmaceutical composition for the treatment of pain, comprising a nucleic acid sequence which is an “antisense” sequence compared to a nucleic acid sequence encoding Cathepsin Y of SEQ ID NO: 2, or SEQ ID NO 4, or homologues or fragments thereof.

10. The pharmaceutical composition of claim 9, wherein said pain is neuropathic pain.


SEQUENCE LISTING (9 sequences)

SEQ ID NO: 1 - DNA, 255 bp, Rattus norvegicus
gggaggagcgaccgacacgaactagaaattatgagcacaagtacactggaatcctccagcttcagagctgcttcctccacccacagacctgcttcctcctccaccgtgcccgagcaggcctaacctccagaccgtcagagaggacagctatggtctaggacagttctggtgttaccctggagtccacgggaggggaactagtccagactgcctgagatgagtaaagtatctggcgtcaccaaaaaaaaaaaaaaa

SEQ ID NO: 2 - DNA, 1387 bp, Rattus norvegicus
ctcactttatctggggtgcggatcgggtcaagaggttgaaggtgctgtgcgtgatccaggatccaagttggcccccggagcaggagcatggcgtcgtcgggatcggtgcagcagctgcggctggtgctgctgatgttgctgctggccggtgcggcacgggccagcctctacttccgcccgggccagacctgctaccgaccccttcacagggaccacctggctctgctggggcgcaggacctatcctcggccacatgagtacctgtccccagcggatctccccaagaactgggactggaggaatgtgaacggtgtcaactatgccagcgtcacgaggaatcagcatatcccacagtactgtggttcttgctgggcccacggcagcaccagtgccttggcagatcgcatcaacatcaagagaaaaggtgcatggccctccaccctgttgtcggtgcagaatgtcatcgactgcggcaatgctggttcttgtgaggggggcaatgaccttccggtgtgggagtatgcccacaagcatggcatccccgatgagacctgcaacaactaccaggccaaggaccaagaatgtgacaagtttaaccagtgtgggacctgcactgaattcaaagagtgccacaccatccagaattataccctctggagagtgggtgactatggctccctgtccgggagggagaagatgatggcagagatctacgccaatggtcccatcagctgcgggataatggcaaccgagaggatgtctaactacactggaggcatctatactgagtaccagaaccaggccattatcaaccacatcatctctgtagccggctggggtgtcagcaatgatggcatcgagtattggattgtccgcaattcatggggcgagccctggggtgagcgaggctggatgagaattgtgaccagcacctataagggagggacaggttccagctacaacctcgccattgaggaggcctgcacatttggggaccccattgtctaggtagatgtccctggaagcaacactgtgaaccatgatgaggaggggtgattatcgacactggatatgtccattcagctagaaacagtggccctcatgtggacacgaggaccagagtgtgggctgcaccccgagaggtgataggaaaggatgagccacaactacactggaaccctccaccttcagacctgcttccctccacccacagacctgcttcctcctccaccgtgccctgcaggcctacctccagccgccagagaggacagctatggtctaggacagttctggtgttaccctggagtccacgggaggggaactagtccagactgcctgagatgagtaaagtatctggcgtcaccaaaaaaaaaaaaaaaaaaaaaaa

SEQ ID NO: 3 - protein, 306 aa, Rattus norvegicus
MASSGSVQQLRLVLLMLLLAGAARASLYFRPGQTCYRPLHRDHLALLGRRTYPRPHEYLSPADLPKNWDWRNVNGVNYASVTRNQHIPQYCGSCWAHGSTSALADRINIKRKGAWPSTLLSVQNVIDCGNAGSCEGGNDLPVWEYAHKHGIPDETCNNYQAKDQECDKFNQCGTCTEFKECHTIQNYTLWRVGDYGSLSGREKMMAEIYANGPISCGIMATERMSNYTGGIYTEYQNQAIINHIISVAGWGVSNDGIEYWIVRNSWGEPWGERGWMRIVTSTYKGGTGSSYNLAIEEACTFGDPIV

SEQ ID NO: 4 - DNA, 1500 bp, Homo sapiens
ggggtcggccgggtgctaggccggggccgaggccgaggccggggcgggatccagagcgggagccggcgcgggatctgggactcggagcgggatccggagcgggacccaggagccggcgcggggccatggcgaggcgcgggccagggtggcggccgcttctgctgctcgtgctgctggcgggcgcggcgcagggcggcctctacttccgccggggacagacctgctaccggcctctgcggggggacgggctggctccgctggggcgcagcacatacccccggcctcatgagtacctgtccccagcggatctgcccaagagctgggactggcgcaatgtggatggtgtcaactatgccagcatcacccggaaccagcacatcccccaatactgcggctcctgctgggcccacgccagcaccagcgctatggcggatcggatcaacatcaagaggaagggagcgtggccctccaccctcctgtccgtgcagaacgtcatcgactgcggtaacgctggctcctgtgaagggggtaatgacctgtccgtgtgggactacgcccaccagcacggcatccctgacgagacctgcaacaactaccaggccaaggaccaggagtgtgacaagtttaaccaatgtgggacatgcaatgaattcaaagagtgccacgccatccggaactacaccctctggagagtgggagactacggctccctctctgggagggagaagatgatggcagaaatctacgcaaatggtcccatcagctgtggaataatggcaacagaaagactggctaactacaccggaggcatctatgccgaataccaggacaccacatatataaaccatgtcgtttccgtggctgggtggggcatcagtgatgggactgagtactggattgtccggaattcatggggtgaaccatggggcgagagaggctggctgaggatcgtgaccagcacctataaggatgggaagggcgccagatacaaccttgccatcgaggagcactgtacatttggggaccccatcgtttaaggccatgtcactagaagcgcagtttaagaaaaggcatggtgacccatgaccagaggggatcctatggttatgtgtgccaggctggctggcaggaactggggtggctatcaatattggatggcgaggacagcgtggtactggctgcgagtgttcctgagagttgaaagtgggatgacttatgacacttgcacagcatggctctgcctcacaatgatgcagtcagccacctggtgaagaagtgacctgcaacacaggaaacgatgggacctcagtcttcttcagcagaggacttgatattttgtatttggcaactgtgggcaataatatggcatttaagaggtgaaagagttcagacttatcaccattcttatgtcactttagaatcaagggtgggggagggagggagggagttggcagtttcaaatcgcccaactgataaataaagtatctggctctgcacgag

SEQ ID NO: 5 - protein, 303 aa, Homo sapiens
MARRGPGWRPLLLLVLLAGAAQGGLYFRRGQTCYRPLRGDGLAPLGRTTYPRPHEYLSPADLPKSWDWRNVDGVNYASITRNQHIPQYCGSCWAHASTSAMADRINIKRKGAWPSTLLSVQNVIDCGNAGSCEGGNDLSVWDYAHQHGIPDETCNNYQAKDQECDKFNQCGTCNEFKECHAIRNYTLWRVGDYGSLSGREKMMAEIYANGPISCGIMATERLANYTGGIYAEYQDTTYILHVVSVAGWGISDGTEYWIVRNSWGEPWGERGWLRIVTSTYKDGKGARYNLAIEEHCTFGDPIV

SEQ ID NO: 6 - protein, 11 aa, Artificial Sequence
  oligopeptide
LFYDQSPTATI

SEQ ID NO: 7 - protein, 12 aa, Artificial Sequence
  oligopeptide
YKRDMVGGVVIA

SEQ ID NO: 8 - protein, 11 aa, Artificial Sequence
  oligopeptide
EGYYGNYGVYA

SEQ ID NO: 9 - protein, 12 aa, Artificial Sequence
  oligopeptide
FFDEPNPGVTIY